(19) United States
(12) Patent Application Publication
(10) Pub. No.: US 2007/0269543 A1
(43) Pub. Date: Nov. 22, 2007

(54) Use of Red Nocardia Rubric Cell Wall Skeleton in the Preparation of Medicament for Treating Cervical Precancerous Lesion

(75) Inventors: Ce Zhang, (US); Yi Zhang, (US); Xiaoming Hong, (US); Guoying Zhang, (US); Jian Zhao, (US)

Correspondence Address: EDWARD W. GOEBEL, JR.;MACDONALD, ILLIG, JONES & BRITTON LLP, 100 STATE STREET, SUITE 700, ERIE, PA, 16507-1498, US

(73) Assignee: Shenyang Sunbellcom Bio-Pharmaceutical Co., Ltd.

(21) Appl. No.: 11/750,529

(22) Filed: May 18, 2007

(30) Foreign Application Priority Data
May 19, 2006 (CN) 2006100826271

(51) Int. Cl. A61K 35/74

(52) U.S. Cl. 424/780

(57) ABSTRACT

The use of Red Nocardia Rubric Cell Wall Skeleton in the preparation of medicaments for treating cervical precancerous lesion is disclosed in the present application.

### FIELD OF THE INVENTION

The invention refers to the use of Red Nocardia Rubric Cell Wall Skeleton in the medicament preparation, especially in the preparation of medicaments for treating cervical precancerous lesion.

### BACKGROUND OF THE INVENTION

Cervical precancerous lesion is a kind of undiagnosable gynecology disease that has an ability of self-limitation on one aspect, but is potential to develop further on another aspect. The transition period of development from cervical precancerous lesion to the cervical cancer is about ten years. Since persisting a long time, there is enough time to check and treat cervical precancerous lesion as early as possible. Especially the patients with light degree of cervical precancerous lesion are expected to be cured.

Cervical precancerous lesion is usually called as squamous intraepithelial lesion of cervix (SZL) or Cervical intraepithelial neoplasia (CIN). The disease is also subdivided as Atypical squamous cells (ASCUS) and atypical glandular cells (AGUS), low squamous intraepithelial lesion (LSIL) and High squamous intraepithelial lesion (HSIL). The CIN can be further classified to three classes as CIN-I, CIN-II and CIN-III.

According to modern medical theory, the development of cervical cancer is related to the infection of Human Papillomavirus (HPV), the interaction of HPV with herpes simplex virus (HSV) and fungus will further promote the pathological change of the cervical epithelial cells and gradually induce the lesion of epithelial cells and tissue into complete canceration.

Cervical precancerous lesion is such a disease that the cervical epithelial cells and the cell tissue change abnormally but have become cancer. The cervical cancer has a series of precancerous lesions whose occurrence and development are a process from quantitative alternation to qualitative alteration and from gradation to mutation. The typical type of cervical precancerous lesion is the atypical hyperplasia of cervical epithelial cells. So the degree of cell tissue lesion is determined by detecting cells and tissue.

There would be about more than 500 thousand people who suffer cervical cancer, and 200 thousand people who die from it all over the world every year. In China there are about more than 150 thousand people who suffer cervical cancer, and 80 thousand who die from it every year. According to the statistics provided by WHO, the death caused by the cervical cancer is in the second position among the death of women died from cancer all over the world and even in the first position in some developing countries. In the 21^{st} century, cervical cancer has been one of the most dangerous diseases affecting the health of the women of the world.

Nowadays, no effective medicament is used clinically with certain curative effect on treating cervical precancerous lesion. So the medicament for treating cervical precancerous lesion is desired for a long time. Different method is applied by different countries for confronting cervical precancerous lesion. A general means that was applied by most countries is to have women at proper age screened, those suffering from cervical precancerous lesion can be found out by cytology detection method in combination with cytohistologic detection method and HPV infection test. For the patients suffering from cervical precancerous lesion, they can be treated by cervical surface damage method or cervical lesion cutting method according to the degree of the lesion. This means can decrease the possibility of development from cervical precancerous lesion to cervical cancer.

The research directions of this field in the world are focused on two aspects: the first, consummating the detecting methods to make it more accurate and convenient; the second, improving and finding the physical and operating methods, such as applying laser, electrical surgery, and cone cutting operation and the like. The researchers in this field put focus on how to damage the cervical surface and how to cut the cervical lesion; in addition, there are some medicaments to be used for treating cervical precancerous lesion.

In China, the treatments of the cervical precancerous lesions are developing. Because people have a panic feeling of cancer, the phenomenon that dealing with patients only by cytological smearing, overdrawing cervical precancerous lesion and over-treatment is widespread. Therefore, taking use of an effective medicament treatment will change over-treatment phenomenon and will be undoubtedly a more scientific, more exact and more humanistic treatment for cervical precancerous lesion.

The goal that gynecological doctors and researchers are pursuing is to decrease the pain suffered by the patient and to keep the physiological function of the women as more as possible. In other words, the physical treatment usually by doctors now is to damage and to cut the ailing uterus; but present Red Nocardia Rubric Cell Wall Skeleton (Nr-cws) treatment is to protect and recover the cervix. Prior treatment method aims to the “disease”; while present method aims to “etiology”. So the present method is a more scientific, more advanced and more humanistic one.

In the prior art, Red Nocardia rubric Cell Wall Skeleton for External Use (Commercial Name: Nikejar) is a kind of non-specific immune regulator. The effective agents of Red Nocardia Rubric Cell Wall Skeleton (Nr-cws) include muramic acid, arabogalactan and mucopeptide, etc, with the ability of regulating the immune function of the body and increasing the activity of T cells, macrophages and NK cells, as well as inducing the production of IFN and IL, and other immune cytokines. At the present time, Nr-cws is mainly used in the treatment of cervical erosion. It also is reported in the art that Red Nocardia Rubric Cell Wall Skeleton can be used for treating infection caused by Herpes simplex virus (HZV), varicella-zoster virus (VZV), HPV and fungus.

Unexpectedly, the inventor of this invention finds that Nr-cws can be used for treating cervical precancerous lesion, settling the problem that cervical precancerous lesion can only be detected, but there is no effective medicaments to treat it, and thus making it more humanistic of the treatment. Furthermore, it is also possible for the present method to realize not only the early finding of the cervical cancer but also the early treatment thereof. The use of this medicament makes it more active of cervical precancerous lesion treatment so as to remove cervical cancer at its early stage.

### SUMMARY OF THE INVENTION

The invention refers to the use of Red Nocardia Rubric Cell Wall Skeleton in the preparation of medicaments for treating cervical precancerous lesion. The cervical precancerous lesions can be classified as ASCUS, AGUS and SZL, atypical hyperplasia of cervical epithelia, wherein the preparation form of the medicament is ointment, cream, plaster, gelatin, lotion, tincture, liniment, oil agent, cataplasm, aerosol, suppository or effervescent tablet, and furthermore, the medicament can be applied topically and directly on surface of the lesion of the cervix. The medicament can be diluted with NS solution into a certain concentration solution for further application. The concentration of present medicament is 0.3 μg/ml˜1402 μg/ml, preferably 30 μg/ml to 240 μg/ml.

There is so much advantages in using medicament prepared from Red Nocardia Rubric Cell Wall Skeleton (Nr-cws) for treating cervical precancerous lesion, which include as follows:
1. Good safety: there is no side effect being observed and uncomfortable feeling being reported during the treatment.
2. Good suitability: topical application such as smearing for topical damage is used so as to avoid systematical reaction in the body.
3. Strong pertinence: the lesion occurs on skin and mucosa, while the medicament is good immunopotentiator when it is applied on these areas, and so the medicament shows strong pertinence.
4. Non-specificity: the medicament has good effect in treating every type of cervical precancerous lesion, such as ASCUS, AGUS and SZL, atypical hyperplasia of cervical epithelia.
5. Affectivity: the effective rate is 80%, and curative rate is 70%.
6. Easy to be used: only disposable medical utensils are needed in the treatment.
7. To cure as early as possible: according to the principles of cervical cancer treatment, the screening of cervical precancerous lesion starts to realize not only early finding, but also early treating for cervical cancer.
8. Easy to be extended: the present medicament does not damage the cervical epithelial tissue and is highly safe and easy to be accepted by the patients.
9.

The effect of present invention medicament is mainly focused on its immune regulation activity. Since the medicament is applied correctly or) the key lesion part for treating the etiology, it can activate the immune system of the lesion part with low immune function, recover and enhance correspondingly the immune function of patients, which is an important factor for achieving the medicament effect.

Cervical precancerous lesion is a kind of reversible disease. Some patients can recover by themselves; and others keep in the original state; but some other patients having the disease continue to develop to be worse. The key factor dominating the developing direction of disease is if the human's immunity system is normal. This further shows that the normal immunity of a human body can clear up the cervical injured epithelial cells and recover the injured cells and tissue little by little. The fact can be provided as evidence in theory to make sure that Nr-cws can be used to treat cervical precancerous lesion.

After acting on the lesion part, the present medicament can rapidly activate the immune system of the body, gather immune cells to the lesion position and increase the ability of macro phagocytes and NK cells to kill and clear up the pathogen and ailing cells. At the same time, it induces the production of IFN and IL, or other immune cytokines which can inhibit the production of infected cells and break down DNA synthesis of damaged or deficient cells, and furthermore to intermit the process of canceration of cervical epidermic tissue to prevent the development of cervical cancer. The present medicament is diluted with NS solution into a certain concentration solution for use. The concentration is in amount of 0.3˜1402 μg/ml, perferably 30 μg/ml to 240 μg/ml.

To show the present medicament effect. Nr-cws has been used in different test including cytological test, animal test and clinical observation respectively. The result shows that the effect of Nr-cws in the treatment of cervical precancerous lesion is reliable.

### BRIEF DESCRIPTION OF DRAWINGS

FIG. 1A is the state of HeLa cell cycle from control group when tested by the flow cytometer, in which the rate of cells in static stage (G0-G1) is 44.44% and the rate of cells in proliferation stage (S+G2−M) is 56.56%.

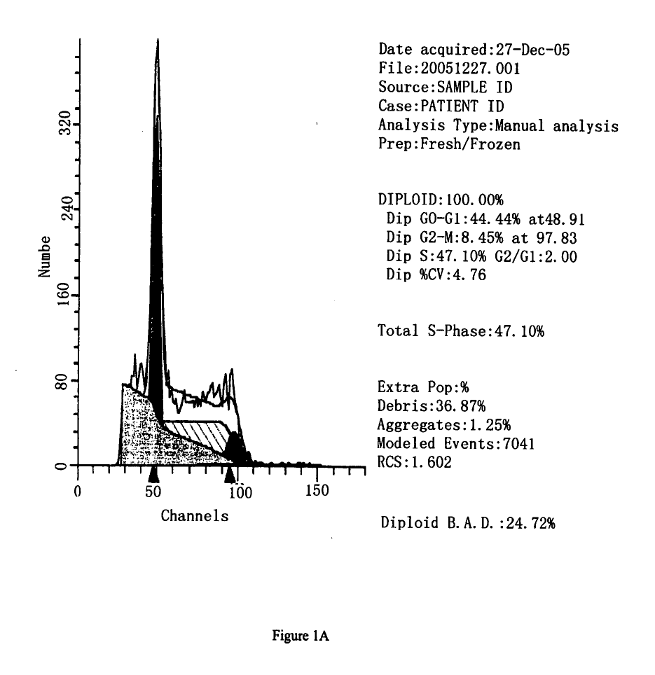

FIG. 1B is the state of HeLa cell cycle from the group applying Nr-cws, in which the rate of cells in static stage (G0-G1) is 74.61% and the rate of cells in proliferation stage (S+G2−M) is 25.39%.

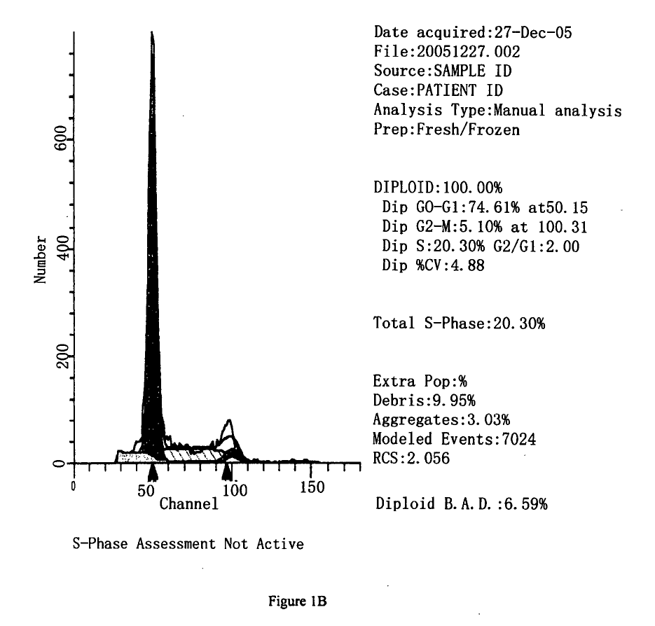

FIG. 2 is HeLa cell growth curve.

### DETAILED DESCRIPTION OF THE INVENTION

The technical solution of the invention will be explained in details by combination with specific examples, just as follows.

HPV infected cervical cancer cells series-Hela cells are tested in groups. Cell growth curve are protracted by analyzing synthetically the vigor of cells detected by MTT of test group applied Nr-cws and blank control group, the result shows: the OD₄₉₀ light absorption value at different time for the two groups are notable different, wherein the growth of the cells is inhibited in the test group; but the growth of the cells is obvious in the control group. Therefore Nr-cws has the activity of inhibiting the HeLa cells growth.

In the experiment testing the ratio of each stage in the cell cycle by flow cytometer, cells that are inhibited in static stage (G0-G1) from test groups are more than that from control group; while the cells in proliferation stage (S+G2−M) from test group are less than that from control group. So Nr-cws has an ability of inhibiting the mitosis of cervical cancer cell, blocking the cancer cells in the G0/G1 stage and prolonging the process of the cancer cells from G1 stage to S stage, and therefore a chance is provided for injured cells to recover and stop damaged cells into DNA synthesizing stage, and to avoid correspondingly deficiency accompanied with DNA synthesis, and finally to prevent the occurrence of cervical cancer.

When the test of inhibiting cancer cells is carried in test animals, the growth of tumor in mouse from experimental group is inhibited; the growth of tumor in mouse from control group is great. There is obvious difference between the two groups, so that Nr-cws has an activity of inhibiting tumor growth.

In the clinical test, Nr-cws is used to treat the patients suffering from ASCUS, AGUS or cervical squamous epithelial lesion. After the treatment perfect results are obtained that Nr-cws has good activity for treating cervical precancerous lesion.

As required, toxicity test of Nr-cws is also carried out to the tested cells and medicament effect test of Nr-cws to the virus infected cells. In the test, African green monkey kidney cells and human embryo lung duple cells are selected as test cells. At the same time the HZV and VZV are applied as the test virus. By the tests the TD₀ and MIC of Nr-cws solution are determined as well.

### EXAMPLE

1. Detecting the inhibiting activity of anti-cancer drug-Nr-cws in inhibiting cervical cancer cells

Method:

MTT (methylthiazolyl tetrazolium) assay is used to test the vigor of cells, protract the cell growth curve, and then analysis the results by using statistics softwere.

(1) Materials and Agents

DMEM culture medium (Gibco, USA), pancreatic protein enzymes (Sigma, USA), MTT (Sigma, USA), embryo ox serum (Hyclone, USA), dimethylsulfoxide (Sigma, USA), 96 well plate (Corning company)

(2) Preparation of MTT

5 mg/ml stock solution with PBS is prepared, followed by filtrating and sterilizing with 0.22 μm filter membrane. The solution can be kept at 4° C. in the shade for one week.

(3) Preparation of Nr-cws

Nr-cws is provided by Shenyang Sun Bell Com Biopharmaceutical Co. Ltd. The collected Nr-cws that are prepared into a certain concentration of suspension is sterilized and suspended under 0.07 Mpa for 15 min for keeping at 4° C.

Cell Growth Curve and the Cell Vigor Test

1) HeLa cells culture: HeLa cells in the log stage are collected by Trypsin digestion. Using DMEM culture solution is to prepare cell suspension, and the cell concentration is adjusted to 5×10⁴/ml. Well-prepared cell suspension is inoculated into the 96-well culture plate, 0.1 ml/well, and 5 wells for test and control group each, wherein Nr-cws (Nikejar, produced by Shenyang Sun Bell Com Biopharmaceutical Co. Ltd.) is added into the test group to the final concentration of 30 μl/ml and is put into the 5% CO₂ culture box (37° C.) for 24-120 h, the culture solution is changed every two days. Cell growth difference between the two groups is tested at 24 h, 48 h, 72 h, 96 h and 120 h during culture via MTT assay.

2) Cell growth Test: according to the known MMT assay, MMT solution at 20 μl/cell (5 mg/ml stock solution) at every detection point (24 h, 48 h, 72 h, 96 h and 120 h) is added, followed by putting them in the CO₂ culture box for 4 hours and discarding the upper part, DMSO, 150 μl/well is added again, and then keeping it in the culture box for 20 min. ELISA meter is used to determine the light absorption value at 590 nm after the MMT is fully dissolved.

2) Protracting HeLa cell grow curve: the values of each light absorption at each time cluding mean value and S are obtained. The time data is taken as x-axis and the light absorption value as y-axis to protract HeLa cell growth curve.

3) Statistics analysis: SPSS 12.0 software is applied to carry couple T-test based on the values obtained from each group.

Results

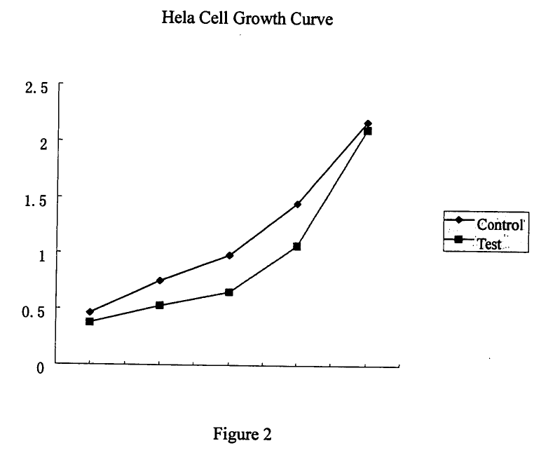

Table-1 is the OD490 light absorption values of HeLa cells at every time and Table-2 is the result of the couple T-test obtained by disposing the values by using SPSS soft statistics analysis, when p<0.05 is shown, it means that there is obvious difference between the two groups. Comparing with HeLa cell growth curve in FIG. 2, test group is obviously different from control group, and Nr-cws can inhibit the growth of the HeLa cells; the tumor cells in test group grow slowly.

TABLE 1
OD490 light absorption values of the HeLa cells at every time point

| | time | | | | |
|---|---|---|---|---|---|
| Group | 24 h | 48 h | 72 h | 96 h | 120 h |
| Control group | 0.46 ± 0.03 | 0.75 ± 0.01 | 0.98 ± 0.05 | 1.45 ± 0.07 | 2.17 ± 0.01 |
| Test group | 0.38 ± 0.02 | 0.53 ± 0.01 | 0.66 ± 0.02 | 1.07 ± 0.1 | 2.10 ± 0.06 |

TABLE 2
Paired Test

| | Paired Differences | | | | | | | |
|---|---|---|---|---|---|---|---|---|
| | | | | 95% Confidence | | | | |
| | | | Std. | Interval of the | | | | |
| | | Std. | Error | Difference | | | | |
| | Mean | Deviation | Mean | Lower | Upper | t | df | Sig. (2-tailed) |
| Pair Control-test | .21600 | .13939 | .06234 | .04292 | .38908 | 3.465 | 4 | .026 |
| group | | | | | | | | |

Flow Cytometer Test

1) Cell culture. HeLa cells to log stage in DMEM containing 10% blood serum are cultured. After digesting it with 0.25% pancreatic enzyme digestion, digested Hela cells are added into 24-well plate at 5000 cells/well to 0.1 ml/well, and 3 wells for test group and control group each. PBS solution is used to wash twice after cells adhering to the wall, followed by culturing in the non-serum culture medium for 12 h to realize synchronization of the cells. The culture medium is exchanged with 10% serum DMEM culture medium to continue curing. Nr-cws is adding into test group to achieve a final concentration of 30 μg/ml. The cells are further cultured in 5% CO2 culture box (37° C.) with changing the solution every two days. Flow cytometer is used to determine the difference of DNA synthesizing between the two groups after one week from the day adding the medicament.

2) Cell preparation: The cells are digested with 0.25% pancreatic enzyme and collected, and then washing them twice with PBS solution. The supernatant is discarded after centrifugation. 0.5 ml cell suspension is kept, and is dispersed by concussion. The cells are injected rapidly into 70% cold ethanol at 4° C. and are kept at the same temperature for at least 18 hours. The cells finally are centrifuged and collected and then RNA enzyme (with a final concentration of 50 μg/ml), containing 0.1% Triton X-100 is added, followed by keeping them in the 37° C. water bath for 30 min and then putting them in the cold ice. The action of RNA enzyme is quenched and washed with PBS solution twice. Propidium iodide (PI) with a final concentration of 50 μg/ml is added and further kept in shade of the cold ice bath for at least 30 min. The cells are filtrated by using 40 μm nylon net before use in the next step.

3) The results: The difference of the DNA synthesis between the test group and the control group are compared. The cells at the stationary stage in the test group are more than that in the control group. The cells at the replicating stage in the test group are less than that in the control group. So it shows that Nr-cws has an activity of inhibiting cervical cancer cells. (Data is shown in the following table)

Analysis on the ratio of every stage during Hela cell proliferation

| Group/stage | (G0-G1) stage | S stage | (G2-M) stage |
|---|---|---|---|
| Control group | 44.44% | 47.1% | 8.45% |
| Test group | 74.61% | 20.30% | 5.1% |

2. Inhibitation of Nr-cws to the tumor cell growth in mouse

| Group/standard | Length*width (cm) | Weight (g) |
|---|---|---|
| Control group (1) | 2.2*1.8 | 2.05 |
| Test group (1) | 1.7*1.2 | 0.7 |
| Control group (2) | 2.4*1.7 | 3.02 |
| Test group (2) | 1.5*0.7 | 0.54 |

Culture density of cell is 10⁶/mouse and the tumor can be seen by our naked eyes after one week (8-9 days).

The medicament (with a concentration of 30 μg/ml) is injected into the tumor in clinical amount, twice/week. The tested mouse is killed after 4 weeks and the length, width and weight of the tumor are measured. The results are showed in the above tables. The growth of the tumor for the test group is slow definitely. The mean weight of tumor for the test group is 0.62 g which is obviously less than that for the control group (2.54 g), having statistic difference.

3. The therapy effect of Nr-cws on clinical patients by local use

The therapy effects of Nr-cws on 10 patients suffering from cervical lesion are showed in table 3. The medicament concentrations for the 10 patients are respectively 30 μg/ml, 60 μg/ml, 120 μg/ml and 240 μg/ml. For the patients, 6 among them are at the age of 28-35, 3 among them are older than 35, one is 26 years old, wherein, 3 patients are suffering from HPV infection meanwhile. After treating with Nr-cws at different dose for different time, 7 patients are cured, 1 patient's condition is relieved and two patients' inflammation is alleviated. For the 3 patients that are not cured, the reason is believed that the condition is very serious and complication is followed, as well as short treatment period. Even is such a disadvantage case, the condition of 3 HPV patients turn negative. When Nr-cws is used for treating cervical precancerous lesion, the curative rate is 70% and effective rate is 80%. So it can be concluded that Nr-cws has a good effect on the treatment of ASCUS, AGUS and atypical hyperplasic lesion. After being treated with Nr-cws the cervical lesion epidermal cells could be cleared up and the injured cervical epithelial tissue could be recovered. Meanwhile, HPV infection can be turned into negative result.

TABLE 3
The observation table of the treatment
for the cervical precancerous lesion

| | | | | Adminis- | Treat- | Post- |
|---|---|---|---|---|---|---|
| Num- | | | Cervical | tration | ment | treatment |
| ber | Name | Age | lesion | way | period | Detection |
| 01 | MAFA* | 36 | ASCUS | Outward, | 2 | Cure |
| | | | | pushing | | |
| 02 | JIHO* | 50 | ASCUS | Outward, | 2 | Cure |
| | | | | pushing, | | |
| | | | | injection | | |
| 03 | WAXU | 30 | AGUS | Outward, | 2 | Cure |
| | | | | pushing | | |
| 04 | LICE | 28 | ASCUS | Outward, | 2 | Cure |
| | | | | pushing | | |
| 05 | XYLI* | 26 | CIN-I | Outward, | 2 | Cure |
| | | | | pushing, | | |
| | | | | injection | | |
| 06 | GANA | 28 | ASCUS | Outward, | 3 | Cure |
| | | | | pushing | | |
| 07 | ZHYE | 35 | Atypical | Outward, | 1 | Relieve the |
| | | | hyper- | pushing | | inflammation |
| | | | plasia, | | | |
| | | | serious | | | |
| | | | erosion | | | |
| 08 | LIXI | 38 | ASCUS, | Outward, | 2 | Relieve the |
| | | | cervical | pushing | | inflammation |
| | | | lesion | | | |
| 09 | LIJI | 34 | CIN-I | Outward, | 2 | Cure |
| | | | | pushing | | |
| 10 | CEMH | 29 | Atypical | Outward, | 1 | Atypical |
| | | | hyper- | pushing | | epithelia |
| | | | plasia | | | |

Noted:
*shows that the patient suffers from HPV infection meanwhile.

4. The medicament effect test of Nr-cws against virus infected cells

1) Toxicity of Nr-cws to the cells:

Nr-cws solution is provided by Shenyang Sunbellcom Biopharmaceutical Co. Ltd. The toxicity of Nr-cws to the cells including VERO cells and 2 BS cells have been observed already.

(1) Toxicity test of Nr-cws solution to 2 BS cells (the results are mean values of three tests).

Tested medicaments: Nr-cws solution CPE method: the maximum nontoxic concentration (TD₀) is 625 μg/ml; median nontoxic concentration (TD₅₀) is 1667 μg/ml. In the MMT method the TD₀ is 625 μg/ml; TD₅₀ is 1417 μg/ml.

(2) Toxicity test of Nr-cws solution to VERO cells (the results are mean values of three tests).

The medicaments: Nr-cws solution CPE method: TD₀ is 1042 μg/ml; TD₅₀ is 2220 μg/ml. In the MMT assay, TD₀ is 958 μg/ml; TD₅₀ is 2376 μg/ml.

2) Inhibitation of Nr-cws to the infected cells:

(1) The inhibitation of Nr-cws solution to HSV-I type virus (the results are mean values of three tests).

\textcircled{1} Group treated with once administration:

Tested medicaments: Nr-cws solution CPE method: the median inhibitory concentration (IC₅₀) is 0.145 μg/ml; maximum inhibitory concentration (MIC) is 0.313 μg/ml; therapeutic index (TI) is 1997. In the MMT method the IC₅₀ is 0.124 μg/ml; MIC is 0.30 μg/ml; TI is 2111.

\textcircled{2} Group treated with three times administration:

Tested medicaments: Nr-cws solution CPE method: IC₅₀ is 0.116 μg/ml; MIC is 0.3 μg/ml; TI is 1997. In the MMT method the IC₅₀ is 0.11 μg/ml; MIC is 0.313 μg/ml; TI is 1997.

(2) Inhabitation of Nr-cws solution to VZV virus (the results are mean values of three tests).

\textcircled{1} Group treated with once administration:

Tested medicaments: Nr-cws solution CPE method; the IC₅₀ is 0.583 μ/ml; MIC is 1.25 μg/ml; TI is 500. In the MMT method the IC₅₀ is 0.456 μg/ml; MIC is 0.846 μg/ml; TI is 779.

\textcircled{2} Group treated with three times administration:

Tested medicaments: Nr-cws solution CPE method: the IC₅₀ is 0.308 μg/ml; MIC is 1.25 μg/ml; TI is 500. In the MMT method the IC₅₀ is 0.25 μg/ml; MIC is 0.342 μg/ml; TI is 1827.

Conclusion of the Test:

\textcircled{1} TD₀ of Nr-cws to the tested cells is: VERO 1042±360 μg/ml, 2BS 625±0 μg/ml.

\textcircled{2} MIC of Nr-cws to the infected cells is:

For group treated with once administration: 0.3±0 μg/ml,

For group treated with three times administration: 0.313±0 μg/ml.

The results are showed in table 4.

TABLE 4
Toxicity test results of Nr-cws solution to VERO and 2BS cells

| | | VERO | | 2BS | |
|---|---|---|---|---|---|
| | | TD0 | TD50 | TD0 | TD50 |
| Medicament | Test method | μg/ml | μg/ml | μg/ml | μg/ml |
| Nr-cws | Change of Cell | 1042 ± 360 | 2220 ± 242 | 625 ± 0 | 1667 ± 0 |
| solution | morpha | | | | |
| | MTT method | 958 ± 0 | 2376 ± 213 | 625 ± 0 | 1417 ± 144.3 |

TABLE 5
Efficiency test of Nr-cws solution against HSV-I type virus and VZV virus.

| | | | Group treated with once | | | Group treated with three | | |
|---|---|---|---|---|---|---|---|---|
| | | | administration | | | times administration | | |
| Virus | Medicament | Method | IC50 | MIC | TI | IC50 | MIC | TI |
| HSV-I | Nr-cws | CPE | 0.145 ± 0.01 | 0.313 ± 0 | 1997 | 0.116 ± 0.01 | 0.313 ± 0 | 1997 |
| | Solution | method | | | | | | |
| | | MTT | 0.124 ± 0.04 | 0.30 ± 0 | 2111 | 0.11 ± 0.01 | 0.313 ± 0 | 1997 |
| | | method | | | | | | |

Noted:
the unit of IC50 and MIC is μg/ml, the results showed in the table are the mean values of three te

What is claimed is:

1. The use of Red Nocardia Rubric Cell Wall Skeleton in the preparation of medicaments for treating cervical precancerous lesion.

2. The use according to claim 1, wherein endive dose of Red Nocardia Rubric Cell Wall Skeleton and pharmaceutical acceptable carriers are included in an anti-HPV medicament.

3. The use according to claim 2, wherein the carriers include excipients.

4. The use according to claim 3, wherein the excipient represents dextran.

5. The use according to claim 1, wherein the medicament is a topical used medicament.

6. The use according to claim 5, wherein the topical used medicaments includes ointment, cream, plaster, gelatin, lotion, tincture, liniment, oil agent, cataplasm, aerosol, suppository and effervescent tablet.

7. The use according to claim 5, wherein the topical used medicament is lotion or liniment.

8. The use according to claim 2, wherein 1 ml or 1 mg medicament contains 0.3˜1402 μg Red Nocardia Rubric Cell Wall Skeleton.
